(19) United States
(12) Patent Application Publication
(10) Pub. No.: US 2020/0318151 A1
(43) Pub. Date: Oct. 8, 2020

(54) METHOD FOR PRODUCING MICROBIAL PROBIOTIC BIOFILMS AND USES THEREOF

(71) Applicant: UNIVERSITA' DEGLI STUDI DI FOGGIA

(72) Inventors: Arcangelo LISO, Perugia (IT); Maria Rosaria CORBO, Foggia (IT); Barbara SPERANZA, Foggia (IT)

(21) Appl. No.: 16/303,989

(22) PCT Filed: May 24, 2017
(86) PCT No.: PCT/IB2017/053055

§ 371 Date: Nov. 21, 2018

(30) Foreign Application Priority Data
May 25, 2016 (IT) 102016000053985

(51) Int. Cl. C12P 39/00; B31B 70/74; C12N 1/20; C12N 11/16; C11D 3/38

(52) U.S. Cl. CPC C12P 39/00; B31B 70/79; C11D 3/381; C12N 11/16; C12N 1/20

(57) ABSTRACT

The present invention relates to a method for producing microbial probiotic biofilms and their uses in the biomedical, industrial, food and environmental field.

The present invention relates to a method for producing microbial probiotic biofilms and their uses in the biomedical, industrial, food and environmental field.

The term biofilm refers to a biologically active matrix of cells and extracellular substances associated with a solid surface or, more precisely, to an association of microorganisms adhered to a surface and trapped inside extracellular polymeric substances (EPS) produced by themselves (Costerton J. W. et al., 1987).

Initially considered a negative phenomenon, it has recently been shown that microbial biofilms may play many “useful” roles such as: biodegradation of toxic compounds and pollutants, bioremediation, toxic effluents treatment, and others (Corbo M. R. et al., 2009). Therefore, through the exploitation of the tendency of microorganisms to adhere to surfaces and/or to spontaneously combine into communities, microbial biofilms can be used for innovative applications in the biomedical, industrial, food, and environmental field.

In the biomedical field, for example, if a biofilm is formed by probiotic microorganisms, it can be used as medical device useful to hinder the development of microorganisms responsible for infections, especially those caused by microorganisms of hospitals, typically resistant to common antibiotic actions.

In fact, as widely demonstrated, in the development of direct and airborne transmission of nosocomial infections, the hospital environment (infection reservoir) in which patients live during the hospital stay and, especially, toilets, play a key role.

A probiotic biofilm left to form ad hoc on toilet surfaces and water closets can reduce the spread of pathogenic species that may harbor thereon.

Other potential applications in the biomedical field are: preparations used in skin lesions for the healing processes, to add antibacterial capacity, the coating of implants and catheters, medical devices applied to the oral cavity which might hinder the growth of bacterial species associated with caries and periodontal disease, the coating of contraceptive devices, such as condoms, that can contribute to the integration of the vaginal flora by hindering the development of infections (e.g. bacterial vaginitis).

Moreover, as regards potential applications in the food industry, biofilms can be used to ensure the hygienic-sanitary safety of food products, as well as an extension of their shelf-life, or even to make some foods functional. Again in the food industry, the formation of biofilms by “useful/probiotic” microorganisms may be stimulated on materials commonly used to package food (plastic films, pellicles, combinations for packaging, paper, etc.) in order to develop an innovative active packaging system.

Although the scientific community is very active in the production of research related to the ability of microorganisms to form biofilms, most studies have focused on biofilm formation by pathogens and/or altering (Enterobacter, Listeria, Micrococcus, Streptococcus, Bacillus and Pseudomonas) and therefore undesired microorganisms (Salo S. et al., 2006; Sharma M. and Anand S. K., 2002; Waak E. et al., 2002).

Although recently it has been shown that certain species of lactic bacteria are able to form biofilms and some of them are capable of performing antimicrobial activity against pathogenic microorganisms (Guillier L. et al., 2008; Guerrieri E. et al., 2009; Speranza B. et al., 2009), no studies have been conducted so far on the tendency to adhesion of “virtuous” microorganisms belonging to the genus Bifidobacterium, although their ability to colonize the gastrointestinal tract in the form of biofilm is recognized and documented. Some research was instead conducted on the possibility of using new methods of sanitation, exploiting the principle of biological competition using probiotic products; however, these studies use different strains than those proposed in the present invention, and in particular they use vegetative cells and spores of Bacillus subtilis, Bacillus megaterium and Bacillus pumilus (Vandini A. et al., 2014).

Other studies describe the formation of biofilms by probiotic strains belonging to the genus Lactobacillus and some of them have also been proposed as a means to control the growth of pathogenic microorganisms (Jones S. and Versalovic J., 2009; Fracchia L. et al., 2010) or yeast (patent application EP 2 186 890 A1).

However, all of these studies propose the use of bio-surfactants and compounds with antimicrobial and anti-inflammatory activity produced in greater quantities by Lactobacilli when growing in sessile form.

The authors of the present invention have developed a probiotic biofilm that exploits the in vivo metabolism of two bacterial strains (Lactobacillus and Bifidobacterium) adhering (and not the substances secreted by them and subsequently recovered and used, as in the prior art) having surprising ability of reducing the load cell of pathogenic species, such as Salmonella enteritidis, Escherichia coli, Listeria monocytogenes, Staphylococcus aureus, among others. Moreover, the probiotic biofilm of the invention is characterized by a special adhesive ability and speed of biofilm formation of the two bacterial strains, selected for their totally unexpected synergistic behavior.

Therefore, the object of the present invention is a method for producing a probiotic biofilm, comprising the following steps:

a) co-inoculation of a combination of at least two probiotic bacterial strains belonging to the genus Bifidobacterium and Lactobacillus at a concentration of at least 10⁶-10⁹ UFC/ml, preferably 10⁸ UFC/ml on the surface of a solid support or into a liquid medium;
b) co-culturing the two probiotic bacterial strains for a time period comprised between 24-72 hours, preferably 48 hours, in the absence (water only) or in the lack (peptone water) of nutrients at a temperature comprised between 12-18° C., preferably 15° C.

Preferably, said combination of probiotic bacterial strains comprises Bifidobacterium infantis (DSM20088) and Lactobacillus reuteri (DSM20016).

According to an alternative embodiment of the present invention, said combination may also comprise one or more other probiotic bacterial strains belonging to the genus Lactobacillus and/or Bifidobacterium.

According to a preferred embodiment of the method of the invention, step a) of inoculum of the combination of probiotic bacterial strains takes place directly on the surface of a solid support consisting of a material selected from glass, steel, ceramic, polymers, paper and resin. Preferably, said polymers are selected from polyvinylchloride (PVC), polyethylene and polypropylene.

Polymer materials may be in the form of films, pellicles, combinations for packaging.

More preferably, said resin belongs to the family of polycarbonates, and even more preferably it is Lexan®.

According to a preferred embodiment of the method of the invention, step a) of co-inoculation of a combination of at least two probiotic bacterial strains takes place at a concentration of at least 10⁸ UFC/ml and step b) of co-culture is carried out for a period of 48 hours at a temperature of 15° C.

Preferably, said combination of probiotic bacterial strains comprises B. infantis (DSM20088) and L. reuteri (DSM20016).

According to a preferred embodiment of step b) of the method of the invention, the co-culture in lack of nutrients takes place with the addition of 1% bacteriological peptone.

A further object of the present invention is a probiotic biofilm obtainable using the method above.

According to a further aspect thereof, the invention is directed to the use of the probiotic biofilm obtained using the method of the invention for the sanitation of surfaces present in hospitals.

Nosocomial infections are in fact an actual and major public health and economical issue. Patients who become ill with a hospital infection represent new sources of contagion, and overcrowding in hospitals, the mixed use of toilets and transfers of patients between wards increase the risk of transmission and acquisition of nosocomial infections.

In addition, the widespread use of antibiotics in hospitals for prevention or for therapy may promote the occurrence of antibiotic-resistant bacterial strains.

Sanitizing procedures are therefore generally aimed at reducing and curbing the proliferation of microorganisms present on the surfaces in hospitals (water closets, bidet, sinks, furniture, etc.).

Currently used disinfectants are chemicals that do not exert a targeted biocidal action, but eliminate all types of microorganisms, with a very short and unstable effect.

In addition, due to problems of resistance developed by numerous pathogenic strains, it is necessary to continuously increase the concentrations and the frequency of application of disinfectants, which are harmful for both humans and for the environment.

If left to form ad hoc on the surfaces to be disinfected (e.g. on water closets), the probiotic biofilm according to the invention could reduce or eliminate the dispersion of urine simultaneously with the elimination of pathogenic species that may harbor therein.

Therefore, detergent solutions based on probiotic microorganisms (mixed with ionic or anionic surfactants and detergents molecules) able to quickly form biofilms on materials such as ceramics, PVC and other resins could represent a revolution in cleaning and disinfection methods and for the industries working in these fields.

Other potential biomedical applications of the probiotic biofilms according to the invention are therapeutic ones, as they could enter into the composition of preparations used to promote wound healing or to prevent skin and orifice infections.

The probiotic biofilms according to the invention may also find application for coating implants and catheters, medical devices applied to the oral cavity (i.e. dental braces, dental bites, etc.), chewing gums or contraceptive devices (i.e. condoms).

The invention also provides indication for the use of the probiotic biofilm according to the invention for use in packaging materials for food use.

In fact, a functional biofilm properly formed on different packaging materials (plastic films, pellicles, oven paper, combinations for packaging) represents an innovative packaging system which on the one hand might be able to guarantee the hygienic-sanitary safety of food products and extend their “shelf life”, and on the other hand might be able to make foods “functional”, now that increasingly health conscious consumers tend to buy high-quality health products.

A further object of the invention is a combination of probiotic bacteria comprising Bifidobacterium infantis (DSM20088) and Lactobacillus reuteri (DSM20016).

Said combination may also comprise one or more other probiotic strains belonging to the genus Lactobacillus and/or Bifidobacterium.

A further object of the present invention is the use of the combination of two strains of probiotic bacterial strains B. infantis (DSM20088) and L. reuteri (DSM20016) for the preparation of a probiotic biofilm.

A further object of the present invention is a detergent solution comprising the probiotic biofilm as an active ingredient, optionally together with one or more additional detergents and/or surfactants.

Finally, an object of the present invention is a food-grade packaging material comprising the probiotic biofilm according to the invention.

The present invention will now be described by way of a non-limiting example in the following experimental part, where it will be detailed with preferred examples.

### EXAMPLE 1: ASSESSMENT OF THE TENDENCY OF PROBIOTIC MICROORGANISMS TO ADHERE TO DIFFERENT SURFACES

In order to assess the tendency of probiotic-like microorganisms (virtuous, nonpathogenic) to adhere to different surfaces, 5 strains of bifidobacteria (strains 1-5), 5 strains of lactobacilli (strains 6-10) and 5 strains of probiotic-like yeast (strains 11-15) were investigated. Glass tiles of AISI316L stainless steel (25 mm×50 mm, 0.5 mm thick) and Lexan® (25 mm×75 mm, 0.5 mm thick) were used as surface samples for biofilm formation, suitably treated to remove any fingerprints, grease or oily substances present on the material.

The tiles were immersed in a 3.5% sodium hypochlorite solution for 15 minutes, washed with distilled water and then transferred to a 0.7% phosphoric acid solution. After 15 minutes, the tiles were rinsed with distilled water and allowed to dry for 24 hours.

For biofilm formation, optimum substrate aliquots (MRS broth, Oxoid) were distributed in containers containing each 5 tiles in a vertical position so as to have the entire surface exposed to the medium. Samples containing glass tiles, stainless steel tiles and tiles made of a particular resin belonging to the polycarbonate family (Lexan®) were prepared, all sterilized in autoclave at 121° C. for 15 minutes.

Each sample was inoculated with the related microorganism to be tested (initial inoculum 10² UFC/ml) and incubated, without stirring, for 5 days at the respective optimum temperature (37° C. for bifidobacteria, 30° C. for lactobacilli, 25° C. for yeasts).

Prior to each test, the inoculum was prepared by revitalizing the strains in the respective culture broths and incubating them at their optimal temperatures for 24 hours. For each microbial target tested, the inoculum was harvested by centrifuging the corresponding pre-culture at 4000 rpm for 10 minutes at 4° C. in an ALC 4239R centrifuge (ALC, Milan, Italy) and re-suspending in sterile physiological solution (9 g/l NaCl) tempered at 4° C.

During the tests, the trend of the microbial population in sessile form was monitored by sampling at specified time intervals. The adhered cell detachment was induced by sonication treatment for 3 minutes at 20 watts. Viable and culturable cells were counted on appropriate media.

The following Table 1 shows the maximum cellular load values reached in stationary phase by the 15 microorganisms tested.

TABLE 1

| STRAINS | A (log CFU/cm²) | | |
|---|---|---|---|
| Bifidobacteria | STEEL | GLASS | LEXAN |
| 1. B. animalis | 4.660 ± 0.057 ^{A}* | 4.557 ± 0.216 ^{A} | 3.841 ± 0.416 ^{A} |
| DSM10140 | | | |
| 2. B. subtilis | 5.093 ± 0.175 ^{B} | 4.684 ± 0.096 ^{A} | 5.010 ± 0.016 ^{B} |
| DSM20096 | | | |
| 3. B. infantis | 6.155 ± 0.235 ^{B} | 5.145 ± 0.001 ^{B} | 4.813 ± 0.421 ^{B} |
| DSM20088 | | | |
| 4. B. longum | 4.797 ± 0.155 ^{A} | 4.371 ± 0.278 ^{A} | 5.237 ± 0.170 ^{B} |
| DSM20219 | | | |
| 5. B. breve | 4.407 ± 0.076 ^{A} | 4.831 ± 0.043 ^{A} | 4.053 ± 0.023 ^{A} |
| DSM20213 | | | |
| Lactobacilli | | | |
| 6. L. plantarum | 6.364 ± 0.152 ^{A} | 5.642 ± 0.137 ^{A} | 5.729 ± 0.140 ^{A} |
| DSM2601 | | | |
| 7. L. casei | 4.083 ± 0.166 ^{B} | 3.692 ± 0.144 ^{B} | 5.108 ± 0.240 ^{B} |
| DSM20011 | | | |
| 8. L. delbrueckii | 6.075 ± 0.311 ^{A} | 5.869 ± 0.400 ^{A} | 4.946 ± 0.200 ^{B} |
| DSM20081 | | | |
| 9. L. paracasei | 6.228 ± 0.288 ^{A} | 6.078 ± 0.311 ^{A} | 5.583 ± 0.300 ^{A} |
| DSM20207 | | | |
| 10. L. reuteri | 6.203 ± 0.259 ^{A} | 5.885 ± 0.318 ^{A} | 6.305 ± 0.210 ^{C} |
| DSM20016 | | | |
| Yeasts | | | |
| 11. Kluyveromyces | No adhesion | No adhesion | No adhesion |
| lactis ATCC8585 | | | |
| 12. S. cerevisiae boulardii | 3.002 ± 0.100 ^{A} | 3.025 ± 0.156 ^{A} | 2.895 ± 0.200 ^{A} |
| ATCC MYA-796 | | | |
| 13. S. cerevisiae | 3.234 ± 0.150 ^{A} | 3.309 ± 0.130^{A} | 3.225 ± 0.250 ^{A} |
| WL21** | | | |
| 14. S. cerevisiae | 4.364 ± 0.230 ^{B} | 4.847 ± 0.250 ^{B} | 4.153 ± 0.116 ^{B} |
| WL40** | | | |
| 15. S. cerevisiae | 3.062 ± 0.200 ^{A} | 3.308 ± 0.300^{A} | 3.890 ± 0.205 ^{B} |
| WL45** | | | |

*Within each genus, the values of the same column with the same superscripts do not differ significantly (p > 0.05)
**Autochthonous yeast probiotic-like strains isolated from wine and belonging to the Department of the Science of Agriculture, Food and Environment - University of Foggia

The data are presented as an average of two repetitions and are accompanied by the standard deviation.

The 5 bifidobacteria tested all guaranteed a good adhesion, with some differences between the tested surfaces.

On steel surfaces, all the strains showed similar behavior in terms of lag phase, adhesion rates and maximum load of adhered cells in stationary phase, with the exception of B. infantis DSM20088 (strain 3), characterized by increased adhesion rate and capable of achieving a higher load of adhering cells in less time (few hours), greater than 6 log CFU/cm². Unlike steel, the analysis of glass-adhesion tests did not reveal any significant differences between the tested microorganisms, as all strains investigated exhibited good adhesion capacity even after a few hours from the inoculum, reaching a maximum load of adhering cells equal to about 4.40-5.14 log CFU/cm². Similar results were recorded for adhesion on Lexan®.

As for the lactobacilli tested, the 5 strains guaranteed good in vitro adhesion to the three surfaces tested. The strains showed similar lag phase and adhesion rates, starting to form biofilm after a few hours from the inoculum and reaching the maximum load of adhering cells on the second incubation day. In particular, higher cellular loads were observed than those observed for bifidobacteria. Yeast strains showed a lower tendency to adhesion than the other microbial targets tested.

In this way, it was possible to identify, among the probiotic microorganisms tested, the strains of B. infantis DSM20088 (strain 3) and L. reuteri DSM20016 (strain 10) as two bacterial strains with good ability to adhere to the tested surfaces.

### EXAMPLE 2: OPTIMIZATION OF CONDITIONS ABLE TO PROMOTE A BETTER FORMATION OF FUNCTIONAL BIOFILMS IN TERMS OF SPEED AND AMOUNT OF ADHERING CELLS

In order to identify the optimal conditions to ensure the maximum adhesion of probiotic microorganisms in terms of the rate of formation and the amount of adhering cells, the optimal conditions (inoculation level and culture conditions) adapted to stimulate greater biofilm formation were identified for the first time.

B. infantis DSM20088 and L. reuteri DSM20016 were used in this step. Lexan® tiles (25 mm×75 mm, 0.5 mm thick) were used as surfaces for the biofilm formation.

In order to assess the ability to form biofilm under suboptimal conditions (nutrient deficiency and low temperature), a first test was conducted in which peptone water aliquots (1% of bacteriological peptone, Oxoid) were distributed in 50 ml plastic tubes containing each 1 tile in vertical position so as to have the entire surface exposed to the medium. Samples were inoculated with the relevant microorganism to be tested (initial inoculum ˜10⁸ CFU/ml) and incubated at 15° C. for 48 hours. The inoculum was prepared as described above. Every hour, the samples were stirred for 10 min at 70 rpm. The trend of the microbial population in sessile form was monitored at specified time intervals, as described above. The tests were conducted in double. Microbial counts were transformed into logarithms before standard averages and deviations were calculated and reported in log CFU/cm².

Both microorganisms tested were able to adhere to the Lexan® surface in the lack of nutrients at 15° C. and under dynamic conditions, reaching a cellular load of about 4.8-5.3 log CFU/cm², already after an hour of contact.

Having ascertained that capacity, a second test was conducted to evaluate the effect of the inoculum level on the tendency to adhesion and the possible presence of interactions.

To this aim, in addition to L. reuteri DSM20016 (L) and B. infantis DSM20088 (B), the yeast strain Saccharomyces cerevisiae boulardii ATCC MYA-796 (SB) was tested.

The inoculum levels were modulated in accordance with a simple centroid; this type of experimental design involves three different variables, each of which can take 3 different levels, identified with code 0 (minimum), 1 (maximum), 0.5 (half of the range considered). As independent variables, the inoculum levels of the three target probiotics investigated were selected.

Table 2 shows both encoded values and those actually tested of the independent variables of the experimental design. To the above centroid combinations, control samples were added in which each microorganism was inoculated individually at the three levels of inoculation tested.

TABLE 2

| | |
|---|---|
| Encoded values | Actual values (log CFU/ml) |

| | L | B | SB | L | B | SB |
|---|---|---|---|---|---|---|
| 1 | 1 | 0 | 0 | 9 | 5 | 3 |
| 2 | 0 | 1 | 0 | 5 | 9 | 3 |
| 3 | 0 | 0 | 1 | 5 | 5 | 7 |
| 4 | 0.50 | 0.50 | 0 | 7 | 7 | 3 |
| 5 | 0.50 | 0 | 0.50 | 7 | 5 | 5 |
| 6 | 0 | 0.50 | 0.50 | 5 | 7 | 5 |

Biofilm formation was favored as described above (suboptimal nutrient deficiency and low temperature conditions).

Samples were inoculated with their microorganisms at the levels indicated in the experimental design. All samples were incubated at 15° C. for 72 hours; every hour, the samples were stirred for 10 min at 70 rpm (with a night stop of about 12 hours).

The trend of the microbial population in sessile form was monitored by sampling at specified time intervals, as already described. Viable and culturable cells were counted on appropriate selective media. The data are presented as an average of two repetitions and accompanied by the standard deviation.

Cell counts were used as inputs for the construction of a polynomial equation in order to evaluate the effects of each variable on the adhesion of each target probiotic tested.

Yeast was not able to adhere to the surfaces under any of the tested conditions. The highest levels of adhesion were observed when at least one of the other two strains (L. reuteri DSM 20016 or B. infantis DSM 20088) was inoculated at the highest level.

As the level of inoculation of each target increased, its tendency to adhesion increased. In addition, the presence of L. reuteri stimulated the adhesion of B. infantis and vice versa. No effect was recorded for yeast.

These results are shown in the following Tables 3 and 4.

TABLE 3
Cellular load in sessile form (log CFU/cm²) observed for
Lactobacillus reuteri DSM 20016 and Bifidobacterium infantis
DSM 20088 after 6 hours of contact with Lexan ®

| | L. reuteri | B. infantis |
|---|---|---|
| | DSM20016 | DSM20088 |
| L + B | 5.689 ± 0.200 | 6.129 ± 0.132 |
| (combination 1) | | |
| L | 5.469 ± 0.108 | — |
| (single inoculum) | | |
| B | — | 2.462 ± 0.230 |
| (single inoculum) | | |
| L + B | 5.543 ± 0.023 | 5.402 ± 0.205 |
| (combination 2) | | |
| L | 2.800 ± 0.145 | — |
| (single inoculum) | | |
| B | — | 4.947 ± 0.015 |
| (single inoculum) | | |

TABLE 4
Standardized effects of the inoculum levels of each of the probiotic targets tested
on the adhesion tendency of the other. The bold data indicates significant effects.

| | Bifidobacterium | Lactobacillus | S. cerevisiae |
|---|---|---|---|
| | infantis | reuteri | boulardii |
| | DSM 20088 | DSM 20016 | ATCC MYA-796 |
| Effect of the | 4.814 ± 0.915 | 4.914 ± 0.915 | 1.103 ± 0.915 |
| inoculum level of | | | |
| L. reuteri | | | |
| DSM 20016 | | | |
| Effect of the | 4.652 ± 0.814 | 5.382 ± 0.814 | 2.083 ± 0.814 |
| inoculum level of | | | |
| B. infantis | | | |
| DSM 20088 | | | |
| Effect of the | −0.322 ± 0.519 | −0.033 ± 0.519 | 1.966 ± 0.519 |
| inoculum level of | | | |
| S. cerevisiae boulardii | | | |
| ATCC MYA-796 | | | |

### EXAMPLE 3: MEASUREMENT OF THE EFFECT OF PROBIOTIC BIOFILMS ON THE DEVELOPMENT OF PATHOGENIC MICROORGANISMS

The activities carried out in Examples 1 and 2 allowed the identification for the first time of a method for the formation of a functional biofilm in which the two probiotic strains to be used (B. infantis DSM20088 and L. reuteri DSM20016) are indicated, but also the optimum conditions to ensure maximum adhesion, in terms of adhesion rate and amount of adhering cells.

Specifically, these optimal conditions were identified in:
absence/lack of nutrients (only water or 1% bacteriological peptone)
low temperature (i.e. 15° C.)
co-inoculation of high concentration strains (˜10⁸ CFU/ml).

Once the method had been developed, in order to evaluate the effect of probiotic biofilms on the development of pathogenic microorganisms, evidence was provided on the growth in sessile form of Listeria monocytogenes, Escherichia coli O157:H7, Staphylococcus aureus and Salmonella enteritidis.

Biofilm formation was favored by simultaneously inoculating the cocktail of identified probiotics (B. infantis DSM20088 and L. reuteri DSM20016, about ˜10⁸ CFU/ml) and the pathogenic target (˜10⁷ CFU/ml) on Lexan® tiles (25 mm×75 mm, 0.5 mm thick), left at room temperature (20° C.) for two hours. After this time interval, the tiles were transferred to aliquots of peptone water (1% bacteriological peptone) and incubated at 15° C. for 48 hours. For each pathogenic target, two samples were set up:
ACTIVE (ATT, sample with adhering probiotics)
CONTROL (CNT, sample without probiotics).

The trend of the microbial population in sessile form was monitored by sampling on selective media at specified time intervals. The data are presented as an average of two repetitions and are accompanied by the standard deviation.

Microbial counts were transformed into logarithms before standard averages and deviations were calculated and reported in log CFU/cm².

The following Table 5 shows the cellular loads in sessile form relating to the targets studied; the data analysis shows how the pathogens studied were able to develop in all samples.

TABLE 5
Cellular loads in sessile form (log CFU/cm²) relating to
Listeria monocytogenes, Escherichia coli O157: H7,
Staphylococcus aureus and Salmonella enteritidis
inoculated in samples ATT and CNT

| | | |
|---|---|---|
| Hours | CNT | ATT |

Listeria monocytogenes

| | | |
|---|---|---|
| 0 | 4.209 ± 0.007 ^{A}* | 3.457 ± 0.115 ^{B} |
| 4 | 4.823 ± 0.125 ^{A} | 3.388 ± 0.200 ^{B} |
| 24 | 4.832 ± 0.132 ^{A} | 4.090 ± 0.102 ^{B} |
| 30 | 5.181 ± 0.250 ^{A} | 4.306 ± 0.108 ^{B} |
| 48 | 4.907 ± 0.005 ^{A} | 4.230 ± 0.102 ^{B} |

Escherichia coli O157: H7

| | | |
|---|---|---|
| 0 | 5.486 ± 0.005 ^{A} | 5.204 ± 0.254 ^{A} |
| 4 | 5.430 ± 0.140 ^{A} | 4.190 ± 0.224 ^{B} |
| 24 | 5.557 ± 0.410 ^{A} | 4.101 ± 0.005 ^{B} |
| 30 | 6.129 ± 0.300 ^{A} | 3.820 ± 0.012 ^{B} |
| 48 | 6.004 ± 0.250 ^{A} | 3.800 ± 0.200 ^{B} |

Staphylococcus aureus

| | | |
|---|---|---|
| 0 | 5.069 ± 0.200 ^{A} | 4.879 ± 0.005 ^{A} |
| 4 | 5.016 ± 0.033 ^{A} | 4.705 ± 0.325 ^{A} |
| 24 | 5.570 ± 0.140 ^{A} | 4.889 ± 0.187 ^{B} |
| 30 | 5.156 ± 0.010 ^{A} | 3.845 ± 0.220 ^{B} |
| 48 | 5.164 ± 0.300 ^{A} | 3.705 ± 0.050 ^{B} |

Salmonella enteritidis

| | | |
|---|---|---|
| 0 | 5.942 ± 0.100 ^{A} | 4.373 ± 0.100 ^{B} |
| 4 | 5.380 ± 0.100 ^{A} | 4.469 ± 0.156 ^{B} |
| 24 | 5.526 ± 0.150 ^{A} | 4.536 ± 0.130 ^{B} |
| 30 | 5.350 ± 0.230 ^{A} | 4.877 ± 0.050 ^{B} |
| 48 | 4.984 ± 0.300 ^{A} | 4.773 ± 0.003 ^{B} |

*Values of the same row with the same superscript do not differ significantly (p > 0.05)

However, cellular loads were always lower in ATT samples (presence of probiotic biofilm, about 6.5-7 log CFU/cm²) compared to the CNT samples (absence of probiotic biofilm), highlighting that the functional biofilm was able to control the growth of all inoculated pathogenic targets. Biofilms were odorless and invisible to the naked eye. For E. coli 0157: H7, there was a significant decrease in cell load compared to control, of more than 1 and 2 logarithmic cycles after 4 and 48 hours of incubation, respectively. For L. monocytogenes, St. aureus and S. enteritidis, cell load reductions ranged from 0.5 to 1.5 logarithmic cycles. Therefore, if left to form ad hoc on surfaces, such as water closets or materials generally used to package foods, it is confirmed that the biofilm proposed can reduce or eliminate the pathogenic species that may be present (present in concentrations far below those tested in the present study) with the possibility of reducing the onset of nosocomial infections and/or ensuring the hygienic-health safety of food products.

### EXAMPLE 4: ASSESSMENT OF PERSISTENCE AND SURVIVAL OF BIOFILMS TO SANITIZING AGENTS AND THEIR FORMATION ON CERAMICS AND PACKAGING MATERIALS

Tests were conducted to evaluate the persistence and survival of functional biofilms to sanitizing agents (step a) and tests to evaluate their formation on the most common packaging materials used in the food packaging industries (polypropylene, PVC, oven paper, paraffin paper and polyethylene films) and on ceramics (step b). In particular, two sanitizing agents (SAN1 and SAN2) were tested on functional biofilms formed on surfaces of Lexan®, as proposed in the present invention (cell load of about 6.5-7 log CFU/cm²). The agents tested were two common disinfectants based on dibenzylketone/ammonium acetate and chlorhexidine, allowed to act for 5 minutes at room temperature.

After contact, the tiles were rinsed to remove the disinfectant residues and placed under suboptimal conditions (nutrition deficiency and 15° C.) for 24 hours to evaluate the ability to reform the biofilm.

The results are shown in the following Table 6.

The two tested sanitizers were able to reduce the cellular load in sessile form but not to completely remove the biofilm, leaving a residual load of about 2.5-3 log CFU/cm². This load allowed the biofilm to restore almost completely after 24 hours (over 5 log CFU/cm²). The results obtained in step b are shown in the following Table 7. The data are presented as an average of two repetitions and are accompanied by the standard deviation.

TABLE 6
Cellular load in sessile form (log CFU/cm²) on the tile after
5 minutes treatment with sanitizing agents and after subsequent
placement under suboptimal conditions for 24 hours

| | SAN1 | SAN2 |
|---|---|---|
| Starting cell load | 6.576 ± 0.313 | 7.134 ± 0.010 |
| Load after treatment with | 2.752 ± 0.230 | 3.080 ± 0.004 |
| sanitizer (5 minutes) | | |
| Load after placement under | 5.559 ± 0.071 | 5.388 ± 0.129 |
| suboptimal conditions for | | |
| 24 hours | | |

TABLE 7
Cellular load in sessile form (log CFU/cm²) observed on ceramics
and common packaging materials used in the food industry

| | Cellular load in sessile form (log CFU/cm²) | | |
|---|---|---|---|
| MATERIALS | 2 hours | 24 hours | 96 hours |
| Polypropylene | 6.644 ± 0.004^{A}* | 6.144 ± 0.483^{A} | 5.883 ± 0.233^{A} |
| PVC | 6.540 ± 0.142^{A} | 5.646 ± 0.103^{A} | 5.866 ± 0.296^{A} |
| Oven paper | 5.774 ± 0.227^{B} | 5.238 ± 0.148^{B} | 5.248 ± 0.060^{B} |
| Paraffin paper | No adhesion | 4.607 ± 0.218^{C} | 4.528 ± 0.134^{C} |
| Polyethylene film | 6.938 ± 0.002^{C} | 6.033 ± 0.376^{A} | 6.544 ± 0.135^{D} |
| Ceramic | 6.855 ±0.198^{C} | 6.237 ± 0.226^{A} | 6.542 ± 0.143^{D} |

*Values of the same column with the same superscript do not differ significantly (p > 0.05)

Table 7 shows that the functional biofilm according to the invention was successfully formed on all the materials tested, recording a higher cellular load in sessile form on polyethylene and on ceramics.

### BIBLIOGRAPHY

Costerton J. W., Geesy G. G., Cheng K. J. 1987. Annual Review of Microbiology, 41: 435-464.
Corbo M. R., Speranza B., Sinigaglia M. 2009. In: Biofilms: Formation, Development and Properties, Ed. Bailey W. C., Nova Science Publishers, pp. 585-594.
Salo S., Ehavald H., Raaska L., Vokk R., Wirtanen G. 2006. LWT—Food Science and Technology, 39: 460-471.
Sharma M., Anand SK. 2002. Food Control, 13: 469-477.
Waak E., Tham W., Danielsson-Tham M. L. 2002. Applied and Environmental Microbiology, 68: 3366-3370.
Guillier L., Stahl V., Hezard B., Notz E., Briandet R. 2008. International Journal of Food Microbiology, 128: 51-57.
Guerrieri E., de Niederhäusern S., Messi P., Sabia C., Iseppi R., Anacarso I., Bondi M. 2009. Food Control, 20: 861-865.
Speranza B., Sinigaglia M., Corbo M. R. 2009. Food Control, 20: 1063-1067.
Vandini A., Antonioli P., Lanzoni L., Camerada M. T., Coccagna M., Branchini A., Platano D., Balbano P. G., Mazzacane S. 2014. In: L'Ospedale, Edicom, pag. 48-62.
Jones S. E, Versalovic J. 2009. BMC Microbiology, 9: 1-9.
Fracchia L., Cavallo M., Allegrone G., Martinotti M. G. 2010. Current research, technology and education topics in applied microbiology and microbial biotechnology, pag. 827-837.

1. A method for producing a probiotic biofilm, comprising the following steps:
a) co-inoculation of a combination of at least two probiotic bacterial strains belonging to the genus Bifidobacterium and Lactobacillus at a concentration comprised between 10⁶-10⁹ CFU/ml on the surface of a solid support pretreated with a culture medium or into a liquid medium;
b) co-culturing the two probiotic bacterial strains for a time period comprised between 24-72 hours in the absence or in the lack of nutrients at a temperature comprised between 12-18° C.

2. The method for producing a probiotic biofilm according to claim 1, wherein said at least two probiotic bacterial strains are Bifidobacterium infantis (DSM20088) and Lactobacillus reuteri (DSM20016).

3. The method for producing a probiotic biofilm according to claim 1, wherein step a) of co-inoculation is carried out on the surface of a solid support made of a material selected from the group consisting of glass, steel, ceramic, polymers, paper and resin.

4. The method for producing a probiotic biofilm according to claim 1, wherein step a) of co-inoculation is carried out at a concentration of 10⁸ CFU/ml and step b) of co-culture is carried out for a period of 48 hours at a temperature of 15° C.

5. The method for producing a probiotic biofilm according to claim 3, wherein said polymers are selected among polyethylene, polyvinyl chloride and polypropylene.

6. The method for producing a probiotic biofilm according to claim 3, wherein said resin is made of polycarbonate, preferably of Lexan®.

7. The method for producing a probiotic biofilm according to claim 1, wherein said co-culture of step b) in lack of nutrients is carried out in the presence of bacteriological peptone 1%.

8. A probiotic film obtainable by the method according to any one of the claims 1-7.

9. A combination of probiotic bacteria comprising Bifidobacterium infantis (DSM20088) and Lactobacillus reuteri (DSM20016).

10. Use of the combination of probiotic bacteria according to claim 9, for the preparation of a probiotic biofilm.

11. A probiotic biofilm characterized in that it comprises the combination of probiotic bacteria according to claim 9, adhered on a solid support or inoculated in a liquid medium.

12. Use of the probiotic biofilm according to claim 8, for sanification or cleaning of the surface of a structure.

13. Use of the probiotic biofilm according to claim 12, wherein said structure is a device selected from the group consisting of catheter, implant, brace, bite or condom.

14. Use of the probiotic biofilm according to claim 12, wherein said structure is selected from the group consisting of sink, bidet, water closet or piece of furniture.

15. Use of the probiotic biofilm according to claim 8, for the application on a packaging material for use in the food industry.

16. Use of the probiotic biofilm according to claim 15, wherein said packaging material is selected from the group consisting of film, pellicle, combinations for packaging or oven paper.

17. A detergent solution comprising the probiotic biofilm according to claim 8 as active ingredient, optionally together with one or more further detergent agent and/or surfactant.

18. A packaging material for use in the food industry comprising the probiotic biofilm according to claim 8.

19. Use of the probiotic biofilm according to claim 11, for sanification or cleaning of the surface of a structure.

20. Use of the probiotic biofilm according to claim 19, wherein said structure is a device selected from the group consisting of catheter, implant, brace, bite or condom.

21. Use of the probiotic biofilm according to claim 19 wherein said structure is selected from the group consisting of sink, bidet, water closet or piece of furniture.

22. Use of the probiotic biofilm according to claim 11, for the application on a packaging material for use in the food industry.

23. Use of the probiotic biofilm according to claim 22, wherein said packaging material is selected from the group consisting of film, pellicle, combinations for packaging or oven paper.

24. A detergent solution comprising the probiotic biofilm according to claim 11 as active ingredient, optionally together with one or more further detergent agent and/or surfactant.

25. A packaging material for use in the food industry comprising the probiotic biofilm according to claim 11.
